(19) United States
(12) Patent Application Publication
(10) Pub. No.: US 2008/0234775 A1
(43) Pub. Date: Sep. 25, 2008

(54) Implantable Heart Stimulator for Enabling Normal Atrio-Ventricular Stimulation Sequence in the Presence of Av-Nodal Interference

(76) Inventors: Hans Schuller, Lund (SE); Anders Lindgren, Taby (SE)

Correspondence Address: SCHIFF HARDIN, LLP;PATENT DEPARTMENT, 6600 SEARS TOWER, CHICAGO, IL, 60606-6473, US

(21) Appl. No.: 11/885,903

(22) PCT Filed: Mar. 10, 2005
(86) PCT No.: PCT/SE2005/000351

§ 371 Date: May 23, 2008

(51) Int. Cl. A61N 1/365; A61N 1/362

(52) U.S. Cl. 607/19; 607/17

(57) ABSTRACT

A heart stimulator for enhancing cardiac performance of a patient suffering from ventricular rhythms that tend to overdrive the patient's atrial rhythm, has sensing circuitry that detects atrial events and ventricular events, and an atrial stimulator that generates and supplies stimulation pulses to the atrium. A timer generates a detection window having a predetermined duration for the ventricular sensing, the detection window starting upon detection of an intrinsic or stimulated atrial event. If an R-wave is sensed from the ventricle during the detection window, a control unit determines that a focus, that pre-depolarizes the ventricles, exists, and the control unit operates the atrial stimulator to increase the atrial stimulation rate for a predetermined number of consecutive heart cycles.

### BACKGROUND OF THE INVENTION

1. Field of the Invention

The present invention generally relates to an implantable heart stimulator. More particularly, the invention relates to the function of a dual chamber pacemaker system aiming to maintain appropriate atrio-ventricular synchrony also in case of AV-nodal overdrive interference, which otherwise could impede the normal atrial contribution to ventricular filling so as to cause an impairment of a patient's hemodynamic conditions.

2. Description of the Prior Art

Implantable cardiac stimulation devices (pacemakers) are well established in the treatment of a variety of heart rhythm disorders. The fundamental purpose of pacemaker treatment is to restore and to maintain normal heart rhythm with regard to appropriate rate, atrio-ventricular sequence and conduction delay (the latter two provided the atria are not in the state of chronic fibrillation). The stimulation provided by cardiac pacemakers is based on the interaction with sensed intrinsic or provoked cardiac activity.

In a healthy heart, the sinoatrial node (SA node) serves as a natural pacemaker, which initiates electrical impulses causing depolarisation of the atria followed by a transmission of the signal to the atrioventricular node (AV node) and subsequent depolarisation of the ventricles after an appropriate PR time interval. Maintenance of atrio-ventricular synchrony is of outmost importance for optimal cardiac performance with regard to cardiac output. Loss of AV synchrony reduces cardiac output by 10 to 30%. It is therefore desirable to provide a dual chamber pacemaker system with appropriate algorithms to maintain or restore normal AV synchrony despite various distortions of the intrinsic cardiac rhythm.

A possible timing disorder might occur if the AV node begins operating too early after an atrial depolarisation is initiated hereby compromising the heart's natural atrio-ventricular delay. The resulting decreased or absent atrial transport carries the risk for significant lowering of cardiac output. Countermeasures against primary accelerated AV nodal rhythms overriding completely the normal SA node or the atrial stimulation rate are in U.S. Pat. No. 6,493,583. The present invention, however, refers to any upcoming nodal activity during an ongoing PV/AV interval, the occurrence of which is not uncommon during physical exercise in patients suffering from SA nodal dysfunction. Insufficient response of the SA node to physical activity is known as chronotropic incompetence and can occur as delayed onset of rate increase, insufficient “total” rate increase or a too accelerated rate decrease after a workload is completed. For treatment of SA nodal dysfunction it is common to implant a dual chamber pacemaker system with rate responsive function. In case the artificial increase in atrial stimulation rate and/or the maintenance is not sufficient or suboptimal, the risk prevails intrinsic nodal activity may arise. The occurrence of accelerated nodal activity during an ongoing PV or AV interval will have a negative impact on the ventricular filling time and decrease stroke volume resulting in less optimal hemodynamic conditions. While sudden onset of AV-nodal rhythm with inhibition of a dual chamber system can be counteracted by means of U.S. Pat. No. 6,493,583, a slow nodal activity increase with occurrence during normal PV and AV intervals will demand a specific algorithm to increase the actual atrial stimulation rate in order to provide the time required for appropriate atrial transport and thus enabling optimal ventricular filling.

Presently there are several algorithms used for the automatic increase in atrial stimulation rate aiming to suppress atrial tachyarrhythmia. These algorithms are based on the detection of supraventricular extra systoles and would not react on ventricular sensed AV nodal activity.

In U.S. Pat. No. 6,493,583 discloses an implantable cardiac stimulation device and method for treating intrinsic ventricular rhythms associated with loss of atrial transport. A rhythm detector is arranged to detect an intrinsic ventricular rhythm lacking in atrial transport when an R-wave detector detects a predetermined number of successive R-waves at a rate below a given rate and when each successive R-wave fails to be preceded by an atrial event, either intrinsic or paced. When this rhythm is detected, a dual chamber pulse generator delivers an atrial pacing pulse to an atrium of the heart prior to each successive R-wave. The atrial pacing pulses are delivered at an AV delay prior to the R-waves and the pacing rate is held constant for a time period. Thereafter, the pacing rate is gradually reduced during a recovery time period until the prevailing base rate is reached or until the end of the recovery time period.

U.S. Pat. No. 6,453,192 relates to a method of detection of ventricular ectopic beats using ventricular electrogram. This is achieved by detecting the time interval, T, between a point Q defined as the onset of the QRS complex and the peak value of R (T=Q*R) during a heart beat cycle and comparing that time with a running mean value calculated from normal heartbeats.

### SUMMARY OF THE INVENTION

An object of the present invention is to prevent intrinsic ventricular contraction to occur too early in relation to the atrial contraction. If ventricular contraction occurs “premature” there is not enough time for the necessary atrial blood volume transport resulting in a lowered stroke volume and decreasing heart minute volume.

The above-described problem may occur if the atrial frequency decreases in relation to the ventricular frequency, or if the ventricular frequency increases in relation to the prevailing atrial frequency.

The decrease of the atrial frequency in relation to the ventricular frequency may be caused by:
1) too slow of a sinus rate during rest (sinus bradycardia).
2) too slow of a sinus rate acceleration during start of work (chronotropic incompetence).
3) no or too slow sinus rate during work (chronotropic incompetence).
4) too fast of a decrease of sinus rate at end of work (chronotropic incompetence).
5) suboptimal atrial stimulation rate increase of a rate responsive pacemaker system, which is sometimes necessary to program in order to avoid unwarranted rate increase due to less specific or improper sensor response, e.g. arm movement without significant workload, etc.

There may be many different reasons to the described problem, e.g. diseases related to the heart and its conduction system or diseases not directly related to the heart, influences from the nervous system, from hormones and/or enzymes (including vasovagal syncope) and/or from medication.

Thus, the general object of the present invention is to achieve an implantable heart stimulator provided with means preventing the PR- and AR-intervals from being too short.

A further object of the present invention aims to automatically increase the atrial stimulation rate to override possible intrinsic nodal rhythm and thus to restore conditions for appropriate AV-sequential contraction pattern.

In particular, the present invention will provide a benefit for patients suffering from chronotropic incompetence, i.e. a state when the rate increase of the sinus node or the paced atrial rate does not meet the physiological requirement, e.g. during periods of physical exercise. Conditions like that open for compensatory rate increase of the AV-node during physical exercise resulting in less optimal hemodynamics due to compromised atrial transport function and decrease in stroke volume, a problem the present invention aims to solve.

Generally this is solved by the present invention by increasing the atrial stimulation rate until the AR interval has reached a preset value.

According to a preferred embodiment the PR interval is monitored and if the PR interval is shorter than 60 ms the pacemaker begins stimulating the atrium with an atrial stimulating rate that slowly increases until the AR interval is at least 100 ms.

The algorithm may be sensitive to crosstalk. Crosstalk prevails when the ventricular detector senses and interprets noise and disturbances or parts of an atrial stimulation as a QRS. If that is the situation the rate will be falsely increased unless crosstalk prevention measurements are taken.

In particular the present invention is achieved by implementing an AV-nodal rate detection window after each atrial event. Preferably, the lengths of the detection window are 100 ms after an atrial stimulation and 60 ms after a sensed P-wave. In case ventricular sensing is accomplished during the detection window the occurrence of a premature depolarisation of the ventricles is considered to exist. That will result in an immediate temporary increase of the atrial stimulation rate by e.g. five min⁻¹ for five consecutive cycles. Continued detection of native R-waves during subsequent detection windows will initiate a further increase of the atrial stimulation rate up to a maximum of preferably 100 min⁻¹. The absence of positive detection during five consecutive cycles initiates a stepwise decrease (preferably five min⁻¹) of the temporary increased atrial stimulation rate.

### BRIEF DESCRIPTION OF THE DRAWINGS

FIG. 1 schematically shows a simplified ECG-strip that illustrates a situation where the present invention is applicable.

FIG. 2 schematically shows a simplified ECG-strip where the present invention is illustrated.

FIG. 3 shows a block diagram of a heart stimulator according to the present invention.

### DESCRIPTION OF THE PREFERRED EMBODIMENTS

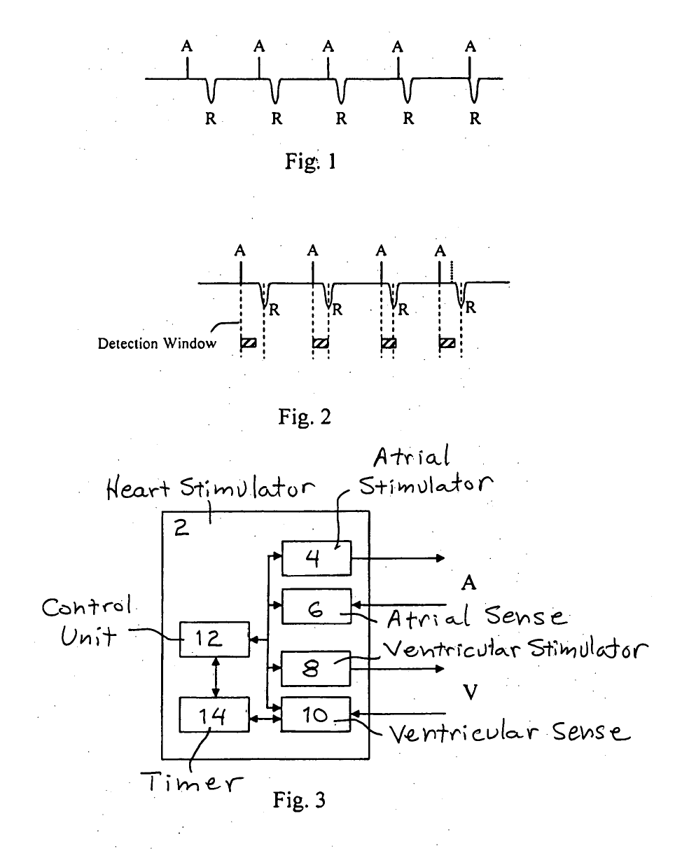

FIG. 1 schematically shows a simplified ECG-strip that illustrates a situation where the present invention is applicable.

FIG. 1 shows five consecutive heart cycles where atrial stimulation pulses A are applied. These pulses are illustrated by vertical bars. The ventricular depolarization is schematically illustrated by a down-going curve designated by an “R”. As is evident from FIG. 1 the time intervals between A and R gradually decreases between the leftmost events to the rightmost events along the horizontal axis. The rightmost events essentially occur at the same time. As discussed above this may result in less optimal hemodynamic conditions.

With references to FIG. 3, the present invention relates to a heart stimulator 2 for enhancing cardiac performance of a patient suffering from ventricular rhythms tending to overdrive the atrial rhythm. The stimulator has a sensing unit 6, 10 for sensing atrial contractions and ventricular contractions, and an atrial stimulator 4 for generating and applying stimulation pulses to the atrium.

The heart stimulator further has a timer 14 for generating a detection window having a predetermined length for said sensing means. The detection window starts upon detection of an atrial depolarisation, intrinsic or stimulated, and if a subsequent ventricular sensing occurs during the ongoing window a “premature” nodal depolarisation of the s the ventricles is considered to exist. This will result in an increase of the atrial stimulation rate for a preset number of consecutive heart cycles.

The present invention provides an additional function to be implemented in dual chamber cardiac stimulation devices aiming for the maintenance of appropriate atrio-ventricular synchrony when “competing” intrinsic nodal activity during an ongoing PV/AV interval occurs.

Preferably, the atrial and ventricular contractions are detected by sensing of the electrical activity of the heart events, the electrogram, related to the depolarization and repolarization of the heart muscle cells.

Alternatively, the atrial and the ventricular contractions may be detected mechanically, e.g. by detecting pressure, movement or sound, or visually, by ultrasound or X-ray techniques.

FIG. 3 shows a block diagram of a heart stimulator according to a preferred embodiment of the present invention.

With references to FIG. 3 the preferred embodiment of the present invention relates to a dual-chamber heart stimulator 2 for enhancing cardiac performance of a patient suffering from ventricular rhythms tending to override the atrial rhythm by shortening the AR interval. The heart stimulator has an atrial sensing unit 6 for sensing P-waves associated with atrial contractions, ventricular sensing unit 10 for sensing R-waves associated with ventricular contractions, an atrial stimulator 4 for generating and applying stimulation pulses to the atrium, and also a ventricular stimulator 8 for generating and applying stimulation pulses to the ventricle.

The heart stimulator further comprises a timer 14 for generating a detection window having a predetermined length for said ventricular sensing means and a control means 12 responsive for the control of all different parts of the heart stimulator. The heart stimulator also comprises (not shown) battery means for power supply of the heart stimulator, at least two heart electrode leads connectable to the heart stimulator and adapted to be inserted into the heart according to established technique, and communication means for performing e.g. bi-directed communication with an external programming unit.

The detection window is started upon detection of an atrial event, intrinsic or stimulated, and if an R-wave is sensed by the ventricular sensing means during the window a focus that pre-depolarizes the ventricles is considered to exist. In that case the atrial stimulation rate is increased for a preset number of consecutive heart cycles.

This situation is schematically illustrated by the simplified ECG-strip in FIG. 2. In FIG. 2, the detection window has lapsed prior the occurrence of the R-wave for the first two heart cycles. In the third heart cycle the R-wave occurs during the detection window and as a consequence the atrial stimulation rate is increased for the next and consecutive heart cycles. This is schematically illustrated by indicating, for the fourth heart cycle, the original position for the stimulation pulse by a dotted vertical line and the applied atrial stimulation pulse at the increased rate by a vertical solid line designated by “A”.

The predetermined detection window length is between 0 and 150 ms, preferably 100 ms, if said atrial event is an atrial stimulation pulse and between 0 and 120 ms, preferably 60 ms, if said atrial event is a sensed P-wave.

According to a preferred embodiment the atrial stimulation rate increase is preferably five stimulation pulses/min during five consecutive heart cycles. The stimulation rate increase may naturally, within the scope of the present invention, be set to a value close to five, e.g. in the interval three to ten stimulation pulses/min in dependence of the prevailing situation.

In a preferred embodiment continued detection of native R-waves during subsequent detection windows initiates a further increase of the atrial stimulation rate up to a maximum rate value, e.g. 100 min⁻¹. This value is variable and set by the physician in relation to the condition of the patient.

The maximum rate value may also be set in dependence of an output signal of an activity sensor (not shown in the figures) such that an increased physical activity of the patient results in an increased maximum rate value.

Thus, according to the present invention if an R-wave is detected in a detection window the atrial stimulation rate is directly increased by e.g. five atrial stimulations per minute during five consecutive heart cycles. If an R-wave still is detected within the detection window and during the five consecutive heart cycles, e.g. in the second of these heart cycles, the rate is then directly further increased.

Absence of native R-wave detections within the detection window during five consecutive heart cycles initiates a stepwise decrease of the increased atrial stimulation rate. Preferably, the stepwise decrease is five stimulation pulses/min in steps of five consecutive heart cycles.

Although modifications and changes may be suggested by those skilled in the art, it is the invention of the inventors to embody within the patent warranted heron all changes and modifications as reasonably and properly come within the scope of their contribution to the art.

We claim as our invention:

1-12. (canceled)

13. A heart stimulator comprising:
sensing circuitry configured to interact with the heart of a patient exhibiting ventricular rhythms tending to overdrive the patient's atrial rhythm, said sensing circuitry sensing atrial contractions and ventricular contractions;
an atrial stimulator configured to interact with an atrium of the heart to generate and apply stimulation pulses to the atrium at an atrial stimulation rate;
a timer connected to said sensing circuitry that generates a detection window having a predetermined duration, said detection window starting upon detection of an intrinsic or stimulated atrial contraction; and
a control unit connected, through said timer, to said sensing circuitry and connected to said atrial simulator, said control unit, if a ventricular contraction is sensed by said sensing circuitry during said detection window, determining that a focus that pre-depolarizes the ventricles exists and, in response, controlling said atrial stimulator to increase the atrial stimulation rate for a predetermined number of consecutive heart cycles.

14. A heart stimulator as claimed in claim 13 comprising a ventricular stimulator configured to interact with at least one ventricle of the heart to stimulate said at least one ventricle, and wherein said sensing circuitry comprises an atrial sensing circuit configured to interact with at least the atrium and a ventricular sensing circuit configured to interact with said at least one ventricle to detect intrinsic or stimulated atrial and ventricular contractions therein, respectively, and wherein said control unit controls said atrial stimulator and said ventricular stimulator dependent on the respective contractions sensed by said atrial sensing circuit and said ventricular sensing circuit.

15. A heart stimulator as claimed in claim 13 wherein said sensing circuitry comprises a mechanical sensor that senses said contractions.

16. A heart stimulator as claimed in claim 15 wherein said mechanical sensor is a pressure sensor.

17. A heart stimulator as claimed in claim 13 wherein said timer sets said duration of said detection window between 0 and 150 ms if said atrial contraction is caused by an atrial stimulation pulse emitted by said atrial stimulator, and between 0 and 120 ms if said atrial contraction is an intrinsic contraction.

18. A heart stimulator as claimed in claim 13 wherein said timer sets said duration of said detection window to be 100 ms if said atrial contraction is caused by an atrial stimulation pulse emitted by said atrial stimulator, and to 60 ms if said atrial contraction is a sensed P-wave.

19. A heart stimulator as claimed in claim 13 wherein said control unit, when a focus that pre-depolarizes the ventricles is determined to exist, increases said atrial stimulation rate by five stimulation pulses/min during five consecutive heart cycles.

20. A heart stimulator as claimed in claim 13 wherein said control unit, after a first of a plurality of said detection windows, operates said atrial stimulator to further increase said stimulation rate up to a maximum rate value, if said sensing circuitry detects intrinsic R-waves during detection windows following said first detection window.

21. A heart stimulator as claimed in claim 20 wherein said control unit employs a rate value of 100 stimulation pulses/min. as said maximum rate value.

22. A heart stimulator as claimed in claim 20 comprising an activity sensor connected to said control unit and configured to detect an indicator of physical activity of the patient, and wherein said control unit increases said maximum rate value dependent on an output of said activity sensor.

23. A heart stimulator as claimed in claim 13 wherein said control unit decreases the increased atrial stimulation rate in steps if said sensing circuitry detects an absence of intrinsic R-waves during five consecutive heart cycles.

24. A heart stimulator as claimed in claim 23 wherein said control unit decreases the increased atrial stimulation rate by five atrial stimulation pulses/min. respectively in five consecutive heart cycles.
